(19) United States
(12) Patent Application Publication
(10) Pub. No.: US 2025/0090424 A1
(43) Pub. Date: Mar. 20, 2025

(54) TRANSFER DEVICE FOR USE WITH INFUSION LIQUID CONTAINER

(71) Applicant: Yukon Medical, LLC, Durham, NC (US)

(72) Inventors: Todd M. Korogi, Durham, NC (US); Gianni Guala, Durham, NC (US); Gonghao Wang, Durham, NC (US); Charles E. McCall, Jr., Durham, NC (US); Jay C. Zignego, Durham, NC (US); Thomas J. Fraites, Jr., Durham, NC (US); Theodore J. Mosler, Durham, NC (US); Stanton D. Batchelor, Durham, NC (US)

(73) Assignee: Yukon Medical, LLC, Durham, NC (US)

(21) Appl. No.: 18/970,254

(22) Filed: Dec. 5, 2024

Related U.S. Application Data
(63) Continuation of application No. 17/431,056, filed on Aug. 13, 2021.
(60) Provisional application No. 62/875,840, filed on Jul. 18, 2019.
(60) Provisional application No. 62/806,478, filed on Feb. 15, 2019.

(51) Int. Cl. A61J 1/20; A61J 1/10

(52) U.S. Cl. CPC A61J 1/2089; A61J 1/10; A61J 1/2013; A61J 1/2062; A61J 1/2075; A61J 1/2082

(57) ABSTRACT

Transfer devices with vial adapter and/or an infusion set for reducing overdosing/underdosing of an infusion bag are provided. The transfer device provides user-controlled fluid transferring between an infusion liquid container and a drug-containing container. In one aspect, the transfer device provides user-controlled venting that can be used during liquid transfer between the infusion liquid container and a drug-containing container.

### CROSS-REFERENCE TO RELATED APPLICATIONS

This application is a continuation of U.S. application Ser. No. 17/431,056, filed Aug. 13, 2021, which is a 35 U.S.C. § 371 National Stage Application of International Application No. PCT/US20/18263, filed Feb. 14, 2020, which claims the benefit of U.S. Provisional Application No. 62/875,840 filed on Jul. 18, 2019, and U.S. Provisional Application No. 62/806,478 filed on Feb. 15, 2019, the entirety of each of which is incorporated herein by reference.

### TECHNICAL FIELD

This disclosure relates to transfer devices for use with infusion liquid container, e.g., intravenous (IV) bags and the like. In one aspect, the transfer device comprises user-controlled fluid transferring between the infusion liquid container and a drug-containing container. In one aspect, the transfer device comprises user-controlled venting that can be used during liquid transfer between the infusion liquid container and a drug-containing container.

### BACKGROUND

Products are available for use with infusion liquid containers containing an infusion liquid and having an intravenous (IV) or administration port. The infusion liquid containers can be in the form of an infusion liquid bag, an infusion liquid bottle, and the like. The transfer devices are also intended for use with additive transfer devices including a male connector and containing a medicine. Conventional transfer devices are also intended for use with infusion sets with an IV spike and a connector. However, such conventional transfer devices are subject to vapor lock, whereby the medicine is incompletely transferred to the infusion liquid containing resulting in a situation where highly concentrated undiluted medicine could be introduced into the infusion line, or, the infusion liquid container is not properly dosed. Either of these situations poses risks and concerns.

### SUMMARY

In a first aspect, a transfer device is provided, the transfer device comprising a three-way connector body having i) a vial adapter coupled to the three-way connector body for receiving a media container with stopper, the vial adapter including a vial spike for fluidic communication therewith, the vial spike comprising at least one vial lumen; and ii) an IV spike integral with the three-way connector body, having a first end for insertion into an infusion liquid container, a first IV lumen, and a second IV lumen adjacent the first IV lumen and in direct fluid communication with the at least one vial lumen. In another aspect, the second lumen is configured for isolated, direct and continuous fluidic communication through the three-way connector body. In yet another aspect, alone or in combination with any one of the previous aspects, the vial adapter is integral with the connector body. In yet another aspect, alone or in combination with any one of the previous aspects, the vial adapter is releasably coupled with the connector body.

In yet another aspect, alone or in combination with any one of the previous aspects, the at least one vial lumen is configurable between a first configuration where the at least one vial spike lumen is in continuous fluid communication with the first IV lumen or second IV lumen; and a second configuration, where, upon rotation of the vial adapter relative to the three-way connector body, the at least one vial spike lumen is in discontinuous fluid communication with the first IV lumen or the second IV lumen.

In yet another aspect, alone or in combination with any one of the previous aspects, the vial spike has at least two physically separated vial spike lumens. In yet another aspect, alone or in combination with any one of the previous aspects, at least one of the at least two physically separated vial lumens is in direct and continuous fluid communication with a vent or fluid filter. In yet another aspect, alone or in combination with any one of the previous aspects, the at least one of the at least two physically separated vial lumens in direct and continuous fluid communication with a one-way check valve. In yet another aspect, alone or in combination with any one of the previous aspects, the one-way check valve is integral with the vial adapter.

In yet another aspect, alone or in combination with any one of the previous aspects, the vial adapter further comprises a hydrophobic filter. In yet another aspect, alone or in combination with any one of the previous aspects, the three-way connector body is coupled with an infusion set. In yet another aspect, alone or in combination with any one of the previous aspects, the IV spike is coupled with an IV spike of another transfer device. In yet another aspect, alone or in combination with any one of the previous aspects, the transfer device further comprises an IV spike adapter coupled to the three-way connector body for receiving an IV set

In another example, a transfer device is provided, the transfer device comprising: a four-way connector body having: i) a first vial adapter coupled to the four-way connector body for receiving a media container with a stopper, the first vial adapter including a first vial spike for fluidic communication therewith, the first vial spike comprising a first fluid lumen; ii) a second vial adapter coupled to the four-way connector body for receiving a media container with a stopper, the second vial adapter including a second vial spike for fluidic communication therewith, the second vial spike comprising a second fluid lumen; iii) an IV spike integral with the four-way connector body, the IV spike having: a first IV lumen in direct fluid communication with the first fluid lumen; a second IV lumen adjacent the first IV spike lumen, the second IV lumen in direct fluid communication with the second fluid lumen; and an infusion lumen providing direct fluid communication through the four-way connector body.

In one aspect, the infusion lumen of the IV spike is configured for isolated, direct and continuous fluidic communication through the four-way connector body.

In another aspect, alone or in combination with any one of the previous aspects, the first vial adapter further comprise a vent body, and the second vial adapter further comprises a second vent body, at least one of the first vent body and the second vent body having a one-way valve or a hydrophobic filter. the first vial spike further comprises a first vent lumen, and the second vial spike further comprises a second vent lumen, wherein the first vent lumen is fluidically coupled with the first vent body and the second vent lumen is fluidically coupled to the second vent body.

In another aspect, alone or in combination with any one of the previous aspects, the first vial adapter and the second vial adapter are integral with the four-way connector body. In another aspect, alone or in combination with any one of the previous aspects, the first vial adapter and the second vial adapter are releasably coupled with the four-way connector body.

In another aspect, alone or in combination with any one of the previous aspects, the transfer device further comprises a cover, the cover transitionable from a hermetically sealed configuration with one of the first or the second vent body to an unsealed configuration.

In another aspect, alone or in combination with any one of the previous aspects, the first vial adapter further comprise a vent body, and the second vial adapter further comprises a second vent body, at least one of the first vent body and the second vent body having a one-way valve or a hydrophobic filter.

In another aspect, alone or in combination with any one of the previous aspects, the device further comprises at least one one-way valve, the at least one one-way valve seated in the vent body.

In another aspect, alone or in combination with any one of the previous aspects, the four-way connector body is coupled with an infusion set. In yet another aspect, alone or in combination with any one of the previous aspects, the transfer device further comprises an IV spike adapter coupled to the four-way connector body for receiving an IV set.

In another aspect, a method of transferring liquid between sealed vials and an infusion liquid container is provided, the method comprising: providing a four-way connector body comprising: a first vial adapter received by the connector body, the first vial adapter comprising: a first vent body; a first spike having a proximal end and a distal end, the proximal end projecting from the first housing, the first spike having a fluid lumen and a vent lumen, the vent lumen in fluidic communication with the vent body. A shroud projects from the first vial adapter and at least partially surrounds a portion of the first spike; the shroud configured to receive a vial or container. The four-way connector body also having a second vial adapter received by the four-way connector body, the second vial adapter comprising: a second vent body; a second spike having a proximal end and a distal end, the proximal end projecting from the second housing, the second spike having a fluid lumen and a vent lumen, the vent lumen in fluidic communication with the vent body. A shroud projects from the second vial adapter and at least partially surrounds a portion of the second spike; the shroud configured to receive a vial or container.

The four-way connector body further comprising at least one cover coupled to one or both of the first vent body or the second vent body and configured to provide a hermetically sealed configuration with the corresponding vent body, the at least one cover transitionable from a hermetically sealed configuration with the corresponding vent body to an unsealed configuration with the corresponding vent body and an IV spike integral with the four-way connector body, the IV spike having an first IV lumen in fluidic communication with the fluid lumen of the first vial adapter, and a second IV lumen adjacent the first IV lumen in direct fluid communication with the fluid lumen of the second vial adapter.

The method further comprising establishing fluidic communication between the sealed vials and the first vial adapter and the second vial adapter, and establishing fluidic communication between the IV spike and the infusion liquid container. The method comprising manipulating the cover of one or both of the first file adapter or the second vial adapter to the unsealed configuration with vent body cover providing venting.

In another example, alone or in combination with any of the previous examples, at least one of the first vent body and the second vent body comprises a one-way valve or a hydrophobic filter.

### BRIEF DESCRIPTION OF DRAWINGS

The present disclosure will now be described, by way of non-limiting examples only, with reference to the accompanying drawings in which similar parts are likewise numbered, and in which:

FIG. 1A is an exploded view of a transfer device as disclosed and described in the present disclosure;

FIG. 1B is a partial exploded view of the transfer device of FIG. 1A;

FIG. 1C depicts a section view of the transfer device of FIG. 1A along line 1C-1C;

FIG. 2A is a side view of a transfer device as disclosed and described in the present disclosure;

FIG. 2B is the opposite side view of the transfer device of FIG. 2A;

FIG. 2C is a top plan view of the transfer device of FIG. 2A;

FIG. 2D depicts a partial section view of the transfer device of FIG. 2B along line 2D-2D;

FIG. 3A depicts a perspective view of a transfer device with fluid access control of a medicament container and an infusion liquid bag;

FIG. 3B depicts an exploded view of the transfer device of FIG. 3A;

FIG. 3C depicts perspective views of the on/off positions, respectfully, of the transfer device of FIG. 3A;

FIG. 3D depicts a section view of the transfer device of FIG. 3A in its off position along line 3D-3D;

FIG. 4 depicts a perspective view of a dual container transfer device as disclosed and described in the present disclosure;

FIG. 5 is an exploded view of the dual container transfer device as disclosed and described in the present disclosure;

FIG. 6A depicts a top view of the dual container transfer device of FIG. 4;

FIG. 6B depicts a section view of the dual container transfer device of FIG. 6A along line 6B-6B;

FIG. 7A depicts a perspective view of the dual container transfer device of FIG. 4 without IV spike and vial container spike covers;

FIG. 7B depicts an exploded top view perspective view of the dual container transfer device of FIG. 4 showing IV spike lumens;

FIG. 8A is a perspective view of a dual container transfer device with user controlled venting as disclosed and described in the present disclosure;

FIG. 8B is a perspective view of a dual container transfer device with one-way valve as disclosed and described in the present disclosure;

FIG. 9 is an exploded view of the dual container transfer device of FIG. 8A, as disclosed and described in the present disclosure;

FIG. 10A depicts a top view of the dual container transfer device of FIG. 8A;

FIG. 10B depicts a section view of the dual container transfer device of FIG. 10A along line 10B-10B;

FIGS. 11A, 11B, and 11C depict exemplary cover assemblies and user controlled cover as disclosed and described in the present disclosure;

FIG. 12 depicts an administration set including an IV bag/infusion liquid bag, an exemplary transfer device as disclosed and described in the present disclosure, a medicament/media vial or container, and an infusion set; and

FIG. 13 depicts an administration set including an IV bag/infusion liquid bag, an exemplary dual container transfer device as disclosed and described in the present disclosure, multiple medicament/media vial or containers, and an infusion set.

FIG. 14 depicts an administration set including an IV bag/infusion liquid bag, an exemplary dual container transfer device with user controlled venting as disclosed and described in the present disclosure, multiple medicament/media vial or containers, and an infusion set.

### DETAILED DESCRIPTION

The present disclosure provides a transfer device that allows effective and substantially complete transfer of medicine or medicament from a medicament container or vial to an infusion liquid container while reducing or eliminating the possibility of medicament remaining in the medicament container or on diluted medicament being presented to the infusion line.

The term “fluid” as used herein, refers to gas, liquid or a combination of gas and liquid. Throughout the specification, the term “liquid” as used herein is inclusive of suspensions, oil-in-water emulsions, water-in-oil emulsions, and liquids with or without dissolved, dispersed, or contained solids irrespective of the size of the solids or the amount present.

As is understood by one having ordinary skill in the art, a fluid transfer device provides for introduction of fluid from one vessel to another, while a fluid control device includes flow control means for diverting, metering, or interrupting flow between at least two flow paths.

Throughout the specification, the term “media” is inclusive of a solid, semi-solid, emulsion, liquid, or combination thereof. For example, the media can be a lyophilized drug, a suspended drug, or an emulsion of a drug.

Throughout the specification, the terms “shroud” and “skirt” are used interchangeably, unless otherwise stated, without any express or implied limitation to the scope of any claim.

Throughout the specification, the phrases “medicament container,” “vial,” “medicament vial/container” and “media container” are used interchangeably, unless otherwise stated, without any express or implied limitation to the scope of any claim.

The following is a description of the drawings and associated features of each aspect of the device and methods disclosed and described herein.

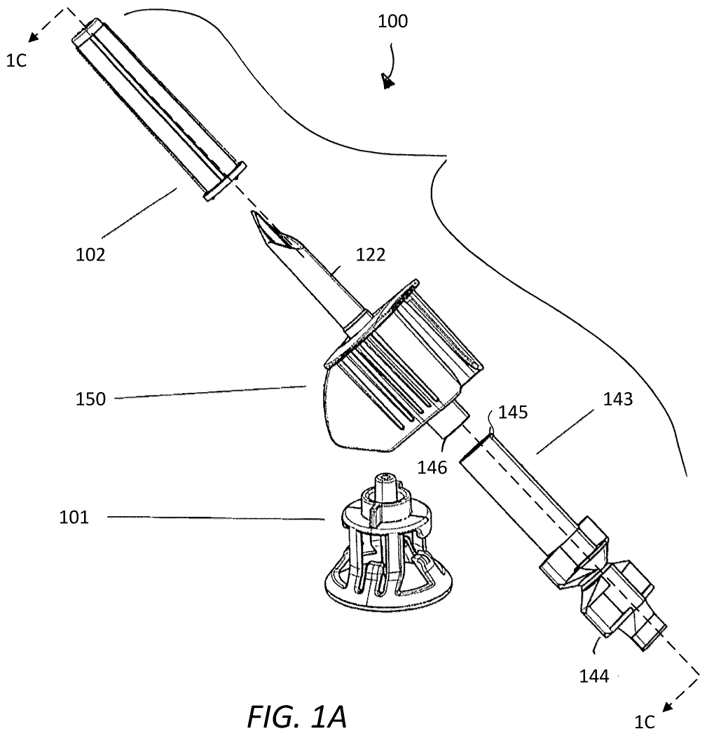

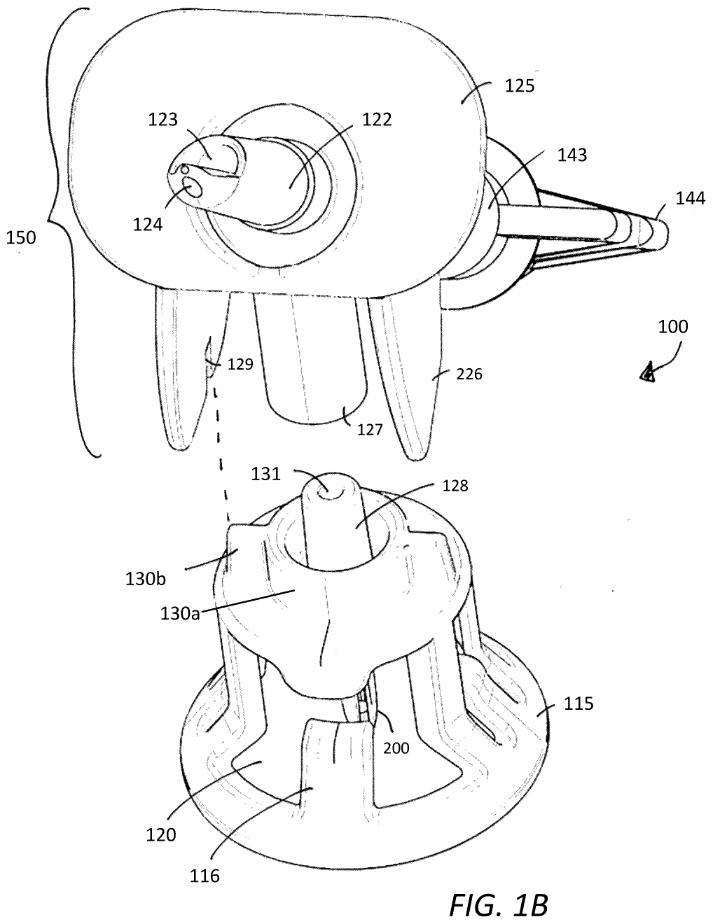

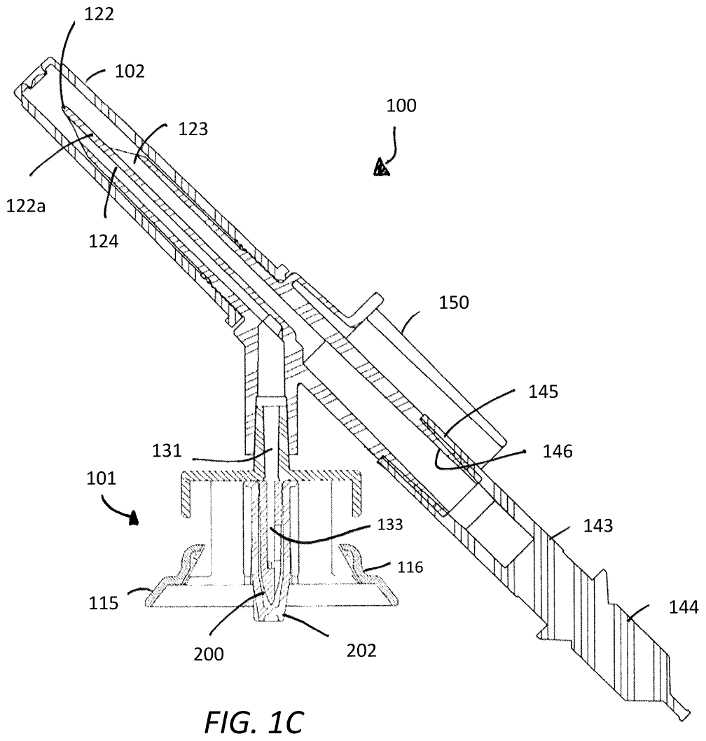

FIGS. 1A, 1B, and 1C depict an exploded view, partial exploded view, and section view along line 1C-1C, respectively, of transfer device 100. The transfer device 100 comprises a three-way connector body 150 configured for coupling with a vial adapter 101. Projecting from the three-way connector body 150 is IV spike 122 shown with removable cap 102. IV spike 122 is configured for insertion into a liquid infusion container and has two lumens 123, 124 physically separated by lumen wall 122a. Projecting from and coupled with an opposite end of the three-way connector body is tapered opening 146 which is received by open end 145 of infusion port 143 that terminates in a twist-off closure member 144.

IV spike 122 projects from surface 125 that provides means for attaching cap 102 and functions to terminate insertion of the IV spike into the liquid infusion container.

Vial adapter 101 contains distal annular edge 115 surrounding vial spike 200 with a single, central fluid lumen 131 and supports the retaining/receiving protrusions or fingers (116). Vial spike 200 is shown with protective cap 202. In one aspect, vial adapter 101 there are four fingers 116 positioned approximately opposite of one another as shown in FIG. 1B with four openings defined by four segments 120 connected the distal annular edge (115). Other shroud configurations can be used. Vial spike 200 is proximately integral to the housing and is open proximal to its distal end for communicating with a vial. Fluid lumen 131 is open proximal to the distal end of the spike or the side of the spike. Spike 200 may be constructed of plastic, metal, or composite material. Spike 200 is designed such that it easily pierces a closure of a vial or drug container, e.g., pointed and/or beveled for facile insertion.

With reference to partial exploded view FIG. 1B and longitudinal cross section of FIG. 1A along line 1C-1C as shown in FIG. 1C, vial adapter 101 includes tapered connector 128 projecting from annual seat 130, connector 128 reversibly receivable by female connector 127 of connector body 150. Connector body 150 all has receiving openings 129 that correspond with wing members 130a of vial adapter 101. Connectors 128 and connector 127 can be permanently coupled e.g. by adhesive, or sonic or solvent welding.

When vial adapter 101 is coupled with connector body 150, tapered connector 128 provides fluidic communication via lumen 131 that is in direct and continuous fluidic communication with lumen 124 of IV spike 122. When vial adapter 101 is coupled with connector body 150, lumen 131 is not in direct and or continuous fluidic communication with lumen 123 of IV spike 122 so as to substantially isolate or prevent fluidic communication therebetween.

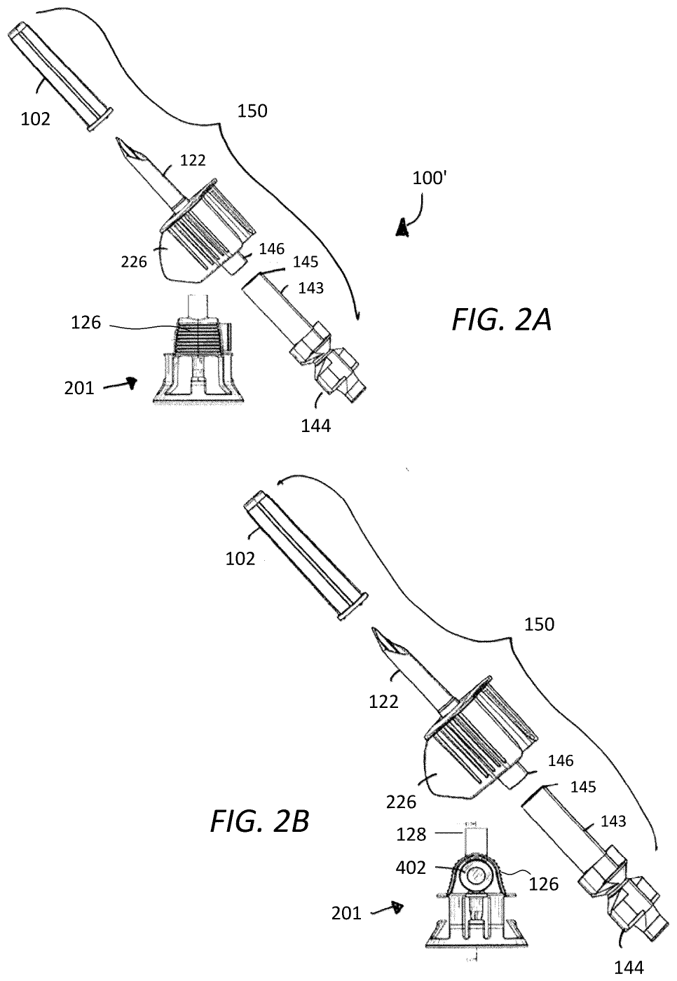

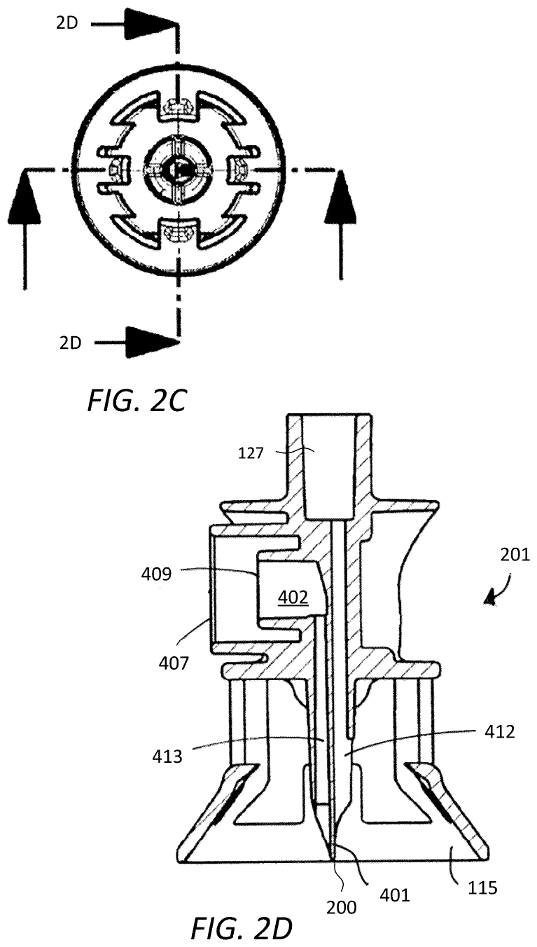

With reference to FIGS. 2A, 2B, that depict side view, opposite side view, top plan view, and partial section view along line 2D-2D, respectively, of transfer device 100′ with vial adapter 201 that has identical connector body 150, IV spike 122, cap 102, and infusion port 143 components but differs from transfer device 100 in that vial adapter 201 includes a vent 402 in vent housing 409 of adapter 201 for one-way communication with the atmosphere. FIG. 2D shows a section view of the vial adapter only, along section line 2D-2D of FIG. 2D. In one example, vent 402 includes a user actuated cover (not shown) described in more detail below. Antimicrobial filter 407 can be employed to filter the air entering device 100′. In one example, antimicrobial filter 407 is hydrophobic, so as to prevent or eliminate liquid from leaving the transfer device 100, 100′.

A vent provides additional safety during use of the disclosed transfer device by relieving pressure that may build up in the fluid and media containers during transfer of liquid volumes of via infusion container squeezing, as well as preventing or eliminating airlock or fluid lock during transfer that otherwise would result in undiluted medicament (overdosing) being introduced into the transfer line, or partially diluted medicament (under dosing) being introduced into the transfer line. In one example, a user controlled vent is provided for venting. In another example, a one-way valve is provided for allowing air to enter the device while preventing or eliminating release of liquid volumes through the device during infusion container squeezing.

Alternatively, one-way fluid communication can be employed by any means capable of restricting fluid flow, such as a check valve. Check valves may be employed as to provide essentially one-way fluid transport through the vent lumen of vial adapter. Check valves may be assembled in a manner that will allow air displaced from the IV bag (during squeezing by user) to vent out of the transfer device to the atmosphere, thus, reducing or eliminating airlock or liquid lock during reconstitution/transfer. The opening may be in communication with a check valve disposed in cooperating relation with the liquid infusion container for providing pressure balance within the transfer device, liquid infusion container, and drug container so as to facilitate effective and substantially complete mixing and/or transferring of the contents of the via to the liquid infusion container. In one example, an air check valve, or a valved air vent and/or a dual lumen vial spike 401 is connected to or integral with vial adapter 201. Exemplary one-way valves include, for example, LOGICA Air valves (Oldenburg, Germany), such as Air valve V2000.

Vial spike 200 is proximately integral to the housing and is open proximal to its distal end for communicating with the vial. Vial spike 200 spike includes at least two lumens, 412, 413, both of which may be open proximal to the distal end of the spike and function independently of each other. The openings in the lumens 412, 413 may be at the distal end of vial spike 200, the side of the spike or one lumen opening may be at the distal end of the spike and another lumen opening may be on the side of the spike. The relative positions of the openings of the lumens 412, 413 proximal to the distal end of vial spike 200 may be the same or different. Vial spike 200 may be constructed of plastic, metal, or composite material. Vial spike 200 is designed such that it easily pierces a closure of a vial or drug container, e.g., pointed and/or beveled for facile insertion.

Vial spike 200 has fluid lumen 412 in direct and continuous fluidic communication with lumen 131, whereas vent lumen 413 is in direct and continuous fluidic communication with vent 402 and is not in direct fluidic communication with lumen 131. Fluid lumen 412 is physically isolated from vent lumen 413 by lumen wall 401, thus, fluidic communication between lumen 412 and lumen 413 is restricted or prevented. Vial adapter 201, in one example, has a tapered male connector 128 that is received by female connector of connector body 150 (not shown). Vial adapter 201, in another example, has a female connector 127 as shown in FIG. 2D, that is received by male connector of connector body 150. Vial adapter 201, in one example, has a tapered male connector 128 that is received by female connector 127 of connector body 150. Gripping surface 126 is shown on vial adapter 201 for a user to grip the vial adapter and parallel features on the surface act as a gripping means.

The presently disclosed transfer device 100′, with a user controlled vented vial adapter and filter, demonstrated that pressure loads against the flow direction had no influence on the function of the valve and/or that wetting of the bacterial filter is effectively prevented by the valve. In contrast, filtered vial adapter, without an air valve or check valve, was found to saturate the filter quickly under pressure, reducing its effectiveness. The air valve (or air check valve) 402 of the presently disclosed transfer device 100′ avoids or eliminates saturation of the filter under pressure.

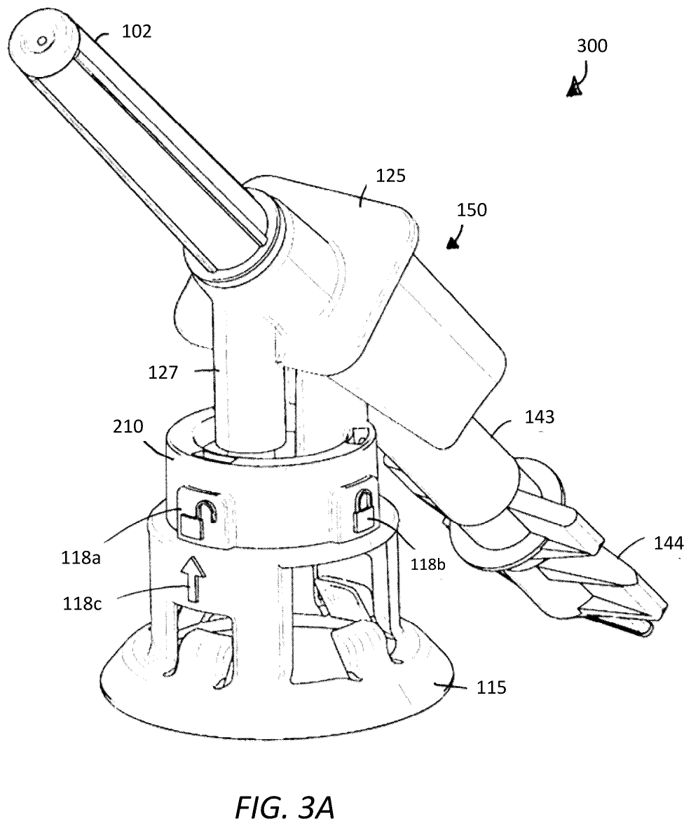

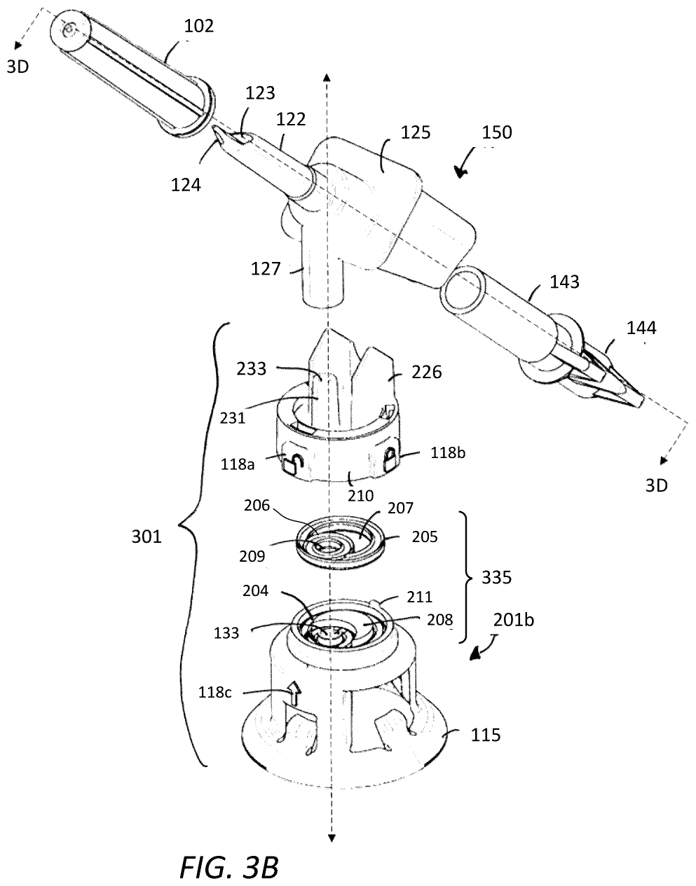

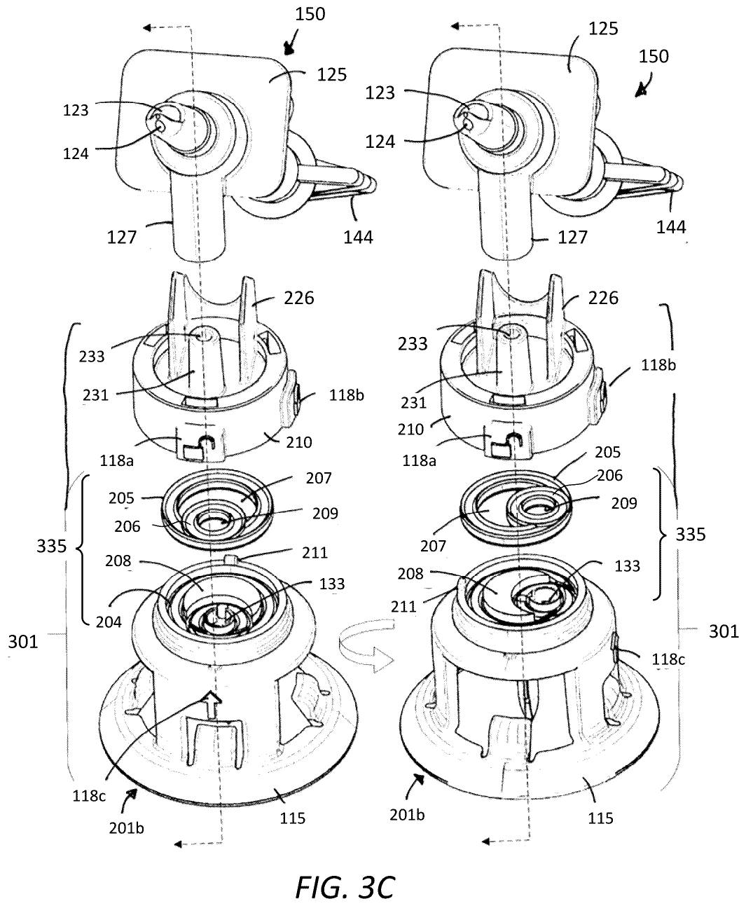

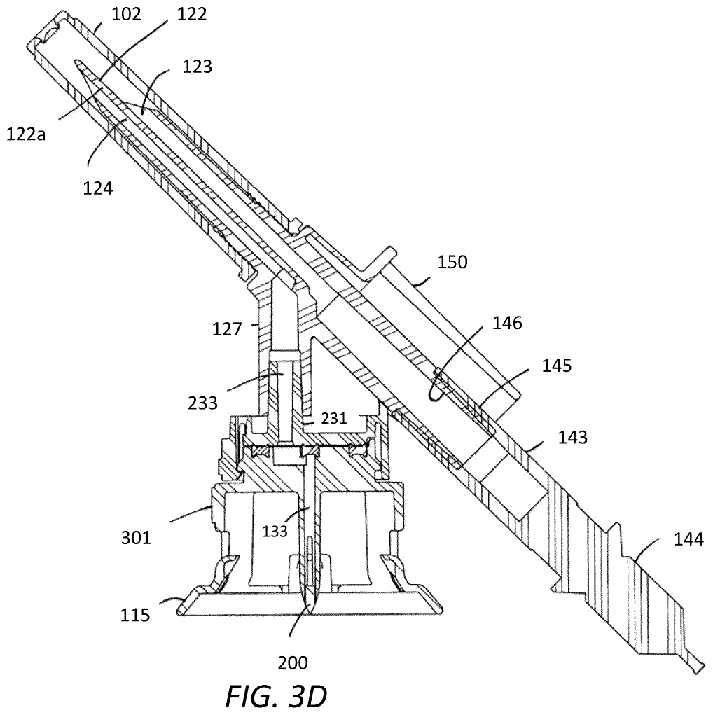

With reference to FIGS. 3A, 3B, 3C, and 3D that depict side view, and exploded views and partial section view along line 3D-3D, respectively, of transfer device 300 with vial adapter 201b that has identical connector body 150, IV spike 122, cap 102, and infusion port 143 components but differs from transfer device 100 and 100′ in that vial adapter 301 includes flow control between vial adapter 201b and lumen 123 of IV spike 122 via female connector 127.

Thus, vial adapter 301, as shown in FIGS. 3A, 3B, 3C, and 3D, provides for controlling fluidic communication to/from the vial. Transfer device 300 as shown provides unvented flow control between a medicament container and the IV spike 122 and/or infusion port 143. Spike 200 is shown configured with fluid lumen 133 with optional offset point. Alternatively, transfer device 300 can be configured to provide flow control in combination with venting by combining the aforementioned attributes of transfer device 100′.

As shown in FIG. 3B, vial adapter 301 includes housing 210 having projecting tapered male connector 231 having fluid pathway 233. Tapered connector 231, partially surrounded by projecting wall 226 of housing 210, is received by projecting female connector 127 of connector body 150. Alternatively, the fluid lumen of the vial adapter can comprise a male or female Luer connection for attaching to a corresponding mating connection of the housing 210 with connector body 150. Other connections can be used, such as threaded connections, e.g. the vial adapter can be replaced with a needleless connector for accessing the fluid path by way of syringe.

Positioned between the stationary housing 210 and rotatable vial adapter 201b is a seal mechanism 335 comprising an outer annular ring 205 and intra-annular ring 206 with opening 209 position between relative stationary housing 210 and rotatable vial adapter 201b. The combination of outer annular ring 205 and intra-annular ring 206 define a crescent shaped opening 207 which aligns with raised crescent surface 208 of rotatable vial adapter 201b. Outer annular ring 205 is received by seat 204 that surrounds raised crescent surface 208 so as to provide a fluidic seal. Intra-annular ring 206 and opening 209 rotatably transitions from a first configuration, where at least one vial spike lumen 133 of vial spike 200 is in continuous fluid communication with lumen 124 of the IV spike 122, to a second configuration, upon rotation of the vial adapter relative to the connector body about the longitudinal axis of the vial spike 200, where the at least one vial spike lumen is in discontinuous fluid communication with lumen 124 of the IV spike 122 to prevent fluid access with fluid pathway of spike lumen 133 of vial spike 200 as shown in FIGS. 3C and 3D. This seal mechanism provides safe fluid control, independent of the shroud position. Pressure loads against the flow direction have substantially no influence on the function of the fluid control of device 300. The seal mechanism is closed in stand-by position. Reflow to reposition the housing 210 is not required.

Indicia (118a, 118b, 118c) is shown placed on rotatable vial adapter 201b as well as the relative stationary housing 210 to assist obtaining the proper positioning of the rotatable vial adapter for choosing control of access of fluid communication between the IV bag/fluid path and the medicament/media container. In one aspect, symbols or colors can be used as the indicia. Other indicia can be used, such as raised or depressed areas. Keyways (211) for orientational control of vial adapter 201b and for preventing release of the flow controller from housing 210 are shown.

### Dual Container Transfer Device

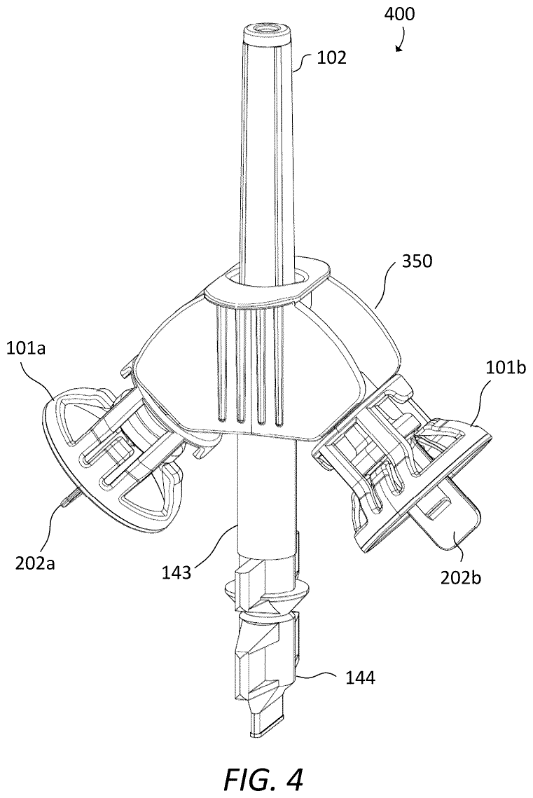

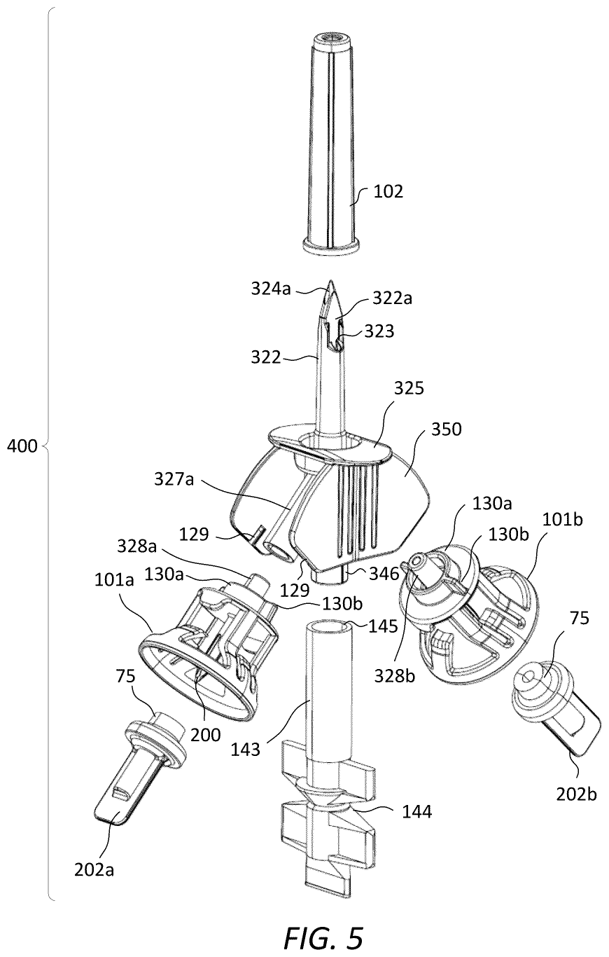

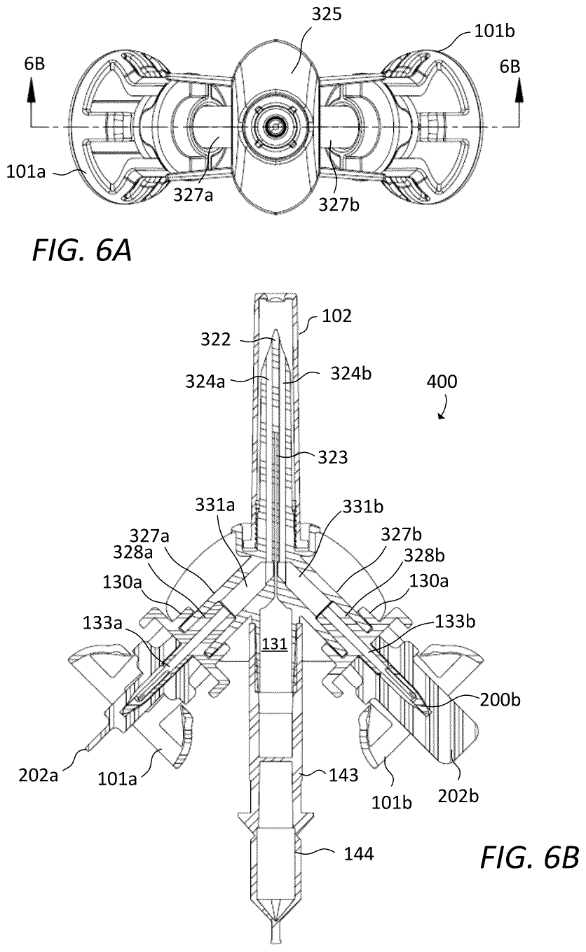

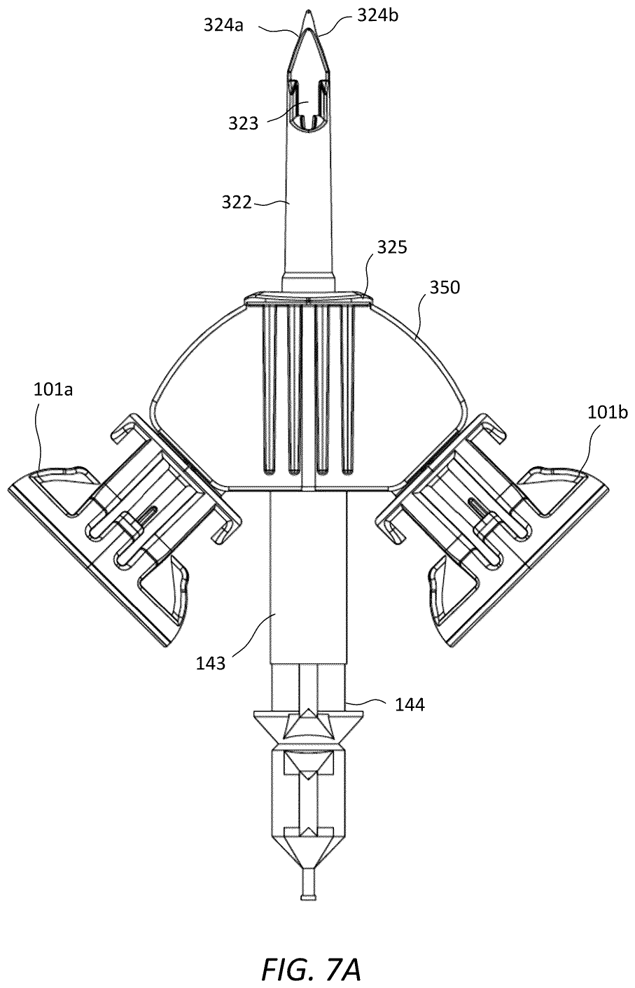

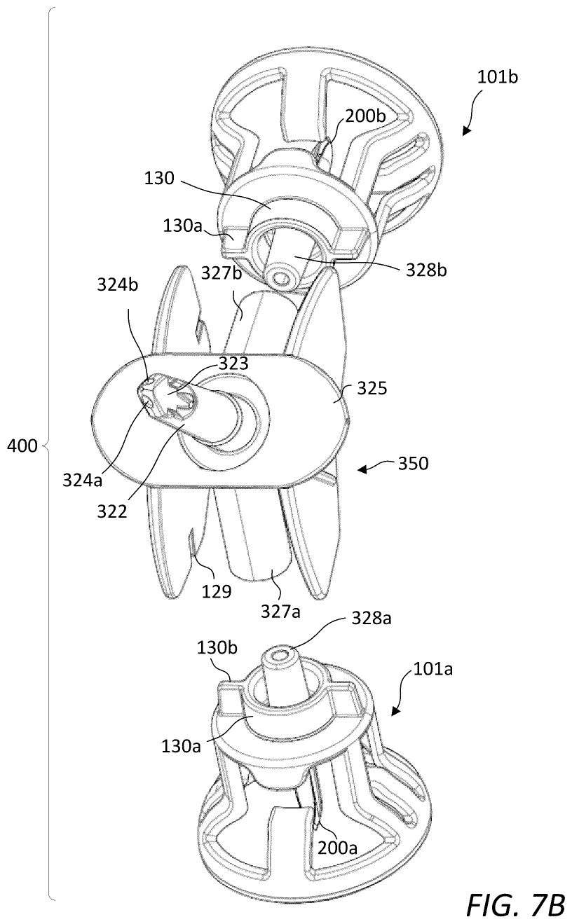

FIG. 4 depicts a perspective view of a dual container transfer device 400. FIG. 5 is an exploded view of the dual container transfer device 400. FIG. 6A depicts a top view of the dual container transfer device 400 and FIG. 6B depicts a section view of the dual container transfer device 400 along line 6B-6B. FIG. 7A depicts a perspective view of the dual container transfer device of FIG. 4 without IV spike and vial container spike covers, and FIG. 7B is an exploded top view of device 400 showing lumens 323, 324a, and 324b of IV spike 322.

As shown in FIGS. 4, 5, 6B and 7A-7B, dual container device 400 is of similar construction as device 100 but for the addition of capability to independently access more than one vial container. In one example, device 400 provides for accessing more than one vial container at the same time.

Thus, device 400 comprises a four-way connector body 350 configured for coupling with two vial adapters 101a, 101b. Coupling of the connector body 350 with each of the two vial adapters 101a, 101b is similar to that of device 100 described above. Projecting from face 325 of the four-way connector body 350 is IV spike 322 shown with removable cap 102, IV spike 322 is configured for insertion into a liquid infusion container and has three lumens 323, 324a and 324b.

Projecting from and coupled with an opposite end of the four-way connector body is tapered opening 346 which is received by open end 145 of infusion port 143 that terminates in a twist-off closure member 144. Lumen 323, along its length from spike 322 distal end to tapered opening 346, is physically separated from lumens 324a and 324b by lumen wall 322a.

Each vial adapter 101a, 101b includes tapered male connector 328a, 328b, respectively reversibly receivable by female connectors 327a, 327b of connector body 350, however, this configuration can be provided with the reverse (female/male) connector. Connector body 350 has receiving openings 129 that correspond with wing members 130b of vial adapters 101a, 101b. Connectors 328a, 328b and connectors 327a, 327b can be permanently coupled e.g. by adhesive, solvent or sonic welding.

When either vial adapter 101a, 101b is coupled with connector body 350, tapered connectors 328a, 328b provides fluidic communication via lumens 331a, 331b that is in direct and continuous fluidic communication with lumen 324a, 324b, respectively, of IV spike 322. Whereas, one vial adapter 101a, 101b is coupled with connector body 350, lumen 131 is not in direct and or continuous fluidic communication with lumen 324a or 324b of IV spike 322 so as to substantially isolate or prevent fluidic communication therebetween.

Lumens 324a and 324b are physically separated from each other along their entire lumen length. Lumen 324a is fluidically coupled to fluid lumen 331a of connector body 350 and lumen 133a of vial adapter 101a. Likewise, Lumen 324b is fluidically coupled to fluid lumen 331b of connector body 350 and lumen 133b of vial adapter 101b. This configuration provides an isolated fluid passage from either of the vial spikes 200 and the corresponding IV fluid lumens 324a, 324b, respectively. One or more vial adapter 101a, 101b, independently, can include a vent and/or check valve for one-way communication with the atmosphere, as described above for device 100′.

### User Controllable Venting

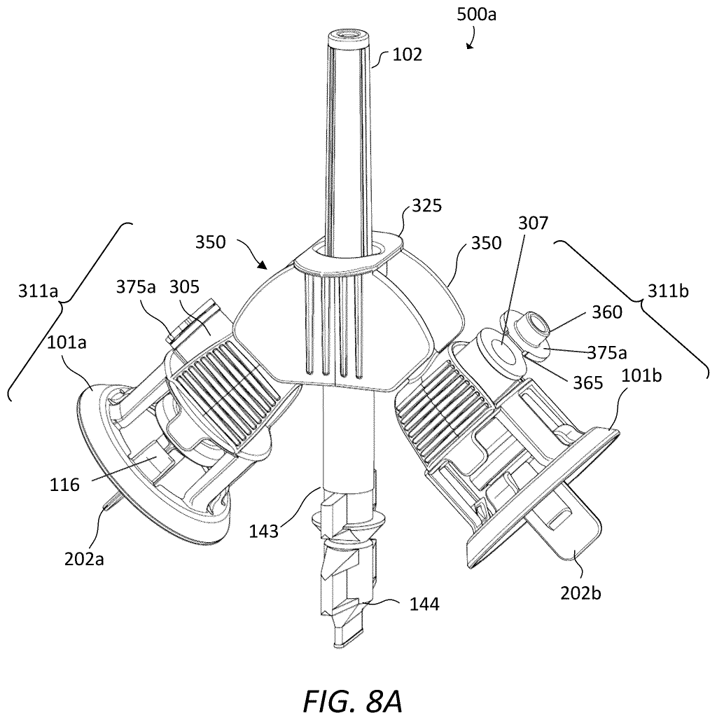

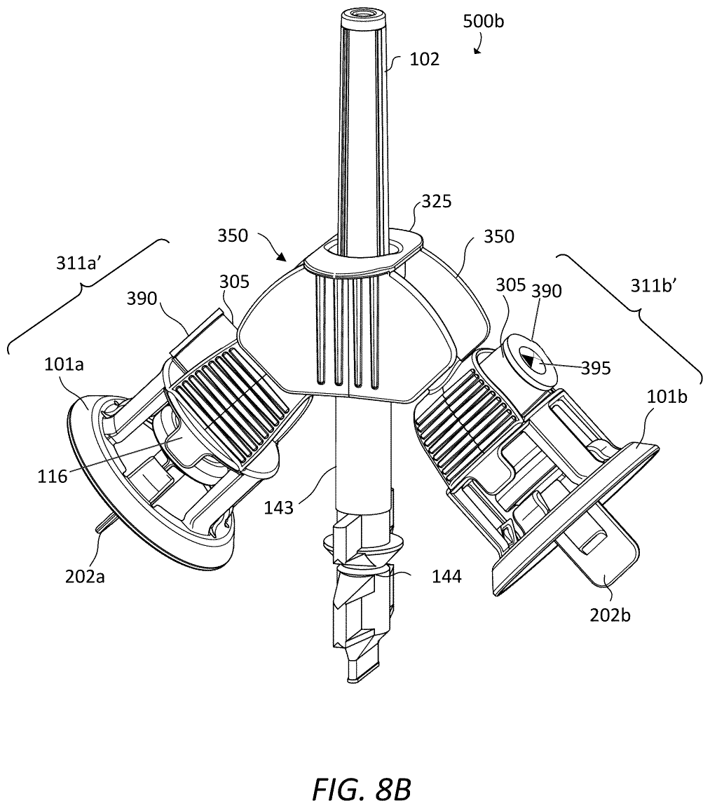

FIGS. 8A through 10B related to devices 500a, 500b having user-controlled venting functionality. In one example, venting is provided with a vent seated in a vent body 305 of housings 311a, 311b and optionally a vent filter, in fluidic communication with a vent lumen of one or more vial spikes 200 where at least one vent body 305 has a user controlled vent cover 375 that can be transitioned from a first state where the vent is closed, to a second state where the vent is open to ambient, discussed below. In another example, venting is provided with a one-way valve vent seated in at least one vent body 305 and optionally, one or both of the one-way valves 395 has a user controlled vent cover 375 that can be transitioned from a first state where the one-way valve is closed, to a second state where the one-way valve is open to ambient, discussed below.

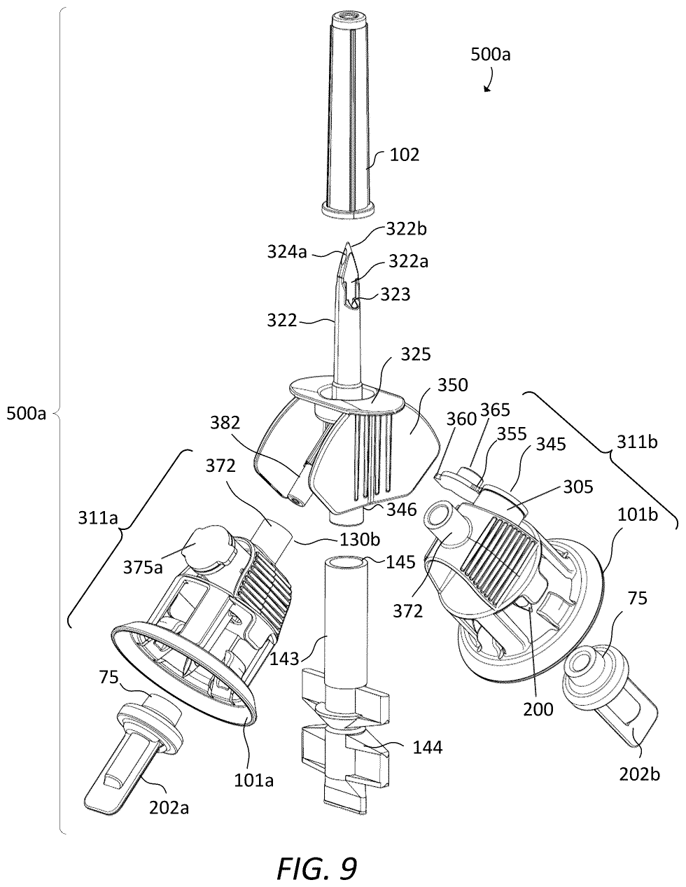

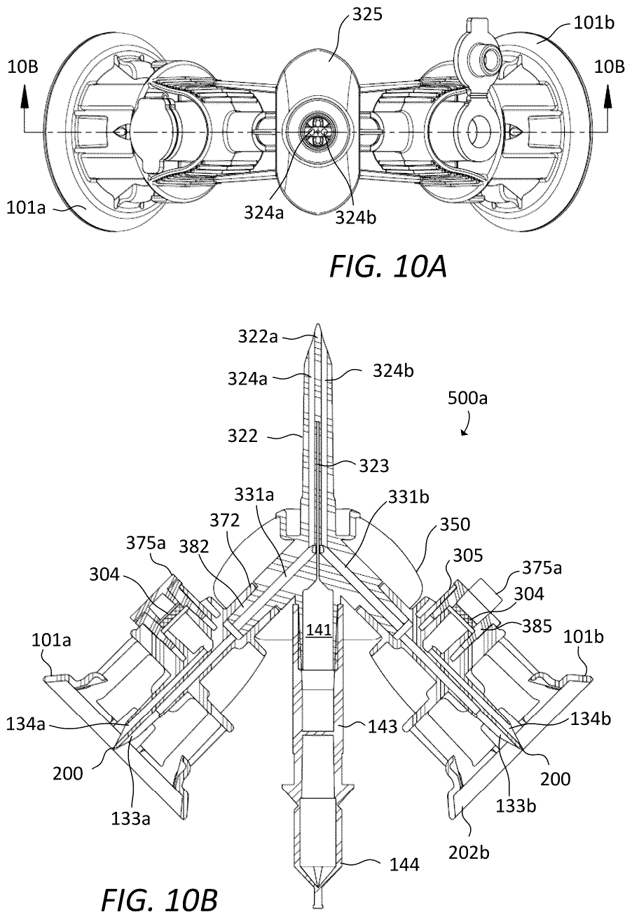

Referring to FIG. 8A, a perspective view of exemplary device 500a is shown in a fully assembled state where the controlled venting is in a first state for vial adapter 101a, and where the controlled venting is in a second state for vial adapter 101b, discussed in more detail below with reference to FIG. 9 showing an exploded view of device 500, and FIGS. 10A-10B. FIG. 10A is a top plan view of device 500 showing section line 10B-10B. FIG. 10B is a sectional view of device 500 alone section line 10B-10B.

Referring to FIG. 8A, a perspective view of exemplary device 500a is shown in a fully assembled state where the controlled venting is in a first state for vial adapter 101a of housing 311a, and where the controlled venting is in a second state for vial adapter 101b of housing 311b, discussed in more detail below with reference to FIG. 9 showing an exploded view of device 500, and FIGS. 10A-10B. Device 500a is similar to device 400 but for a controllable vent cover 375 coupled to one of the vent bodies 305 of housings 311a and 311b. In one example, controllable vent cover 375 (not shown) is coupled to one of vent bodies 305 of housings 311a and 311b.

Referring to FIG. 8B, a perspective view of exemplary device 500b is shown in a fully assembled state where the controlled venting is provided by a one-way valve 395 seated in vent body 305. Device 500b is similar to device 500a but for a one-way valve 395 seated in vent body 305 of one or both of housings 311a′ and 311b′. Device 500b is shown without cover 375, however, cover 375 can be employed as described for device 500a. Device 500b can have one-way valve 395 and controllable vent cover 375 (not shown). In another example, device 500b can comprise controllable vent cover 375 is coupled to one of vent bodies 305 of housing 311a′ and one-way valve 395 is coupled to vent body 305 of housing 311b′.

In one example, a controllable vent cover 375 (not shown) is coupled to one or both of vent bodies 305 of housings 311a and 311b providing for covering one-way valve 395 and preventing venting. In one example, a controllable vent cover 375 is coupled to both vent bodies 305 of housings 311a and 311b providing for closing both one-way valves 395. While not shown, device 500a or 500b can be configured with one vent body 305 with a one-way valve 395 and the other vent body with just a vent filter with controllable vent cover 375. While not shown, alternatively, device 500a or 500b can be configured with one vent body 305 having a one-way valve 395 with a controllable vent cover 375 and the other vent body having a vent filter with controllable vent cover 375.

With reference to FIGS. 9, 10A and 10B, device 500a is shown, comprising a four-way connector body 350 configured for coupling with two vial adapters 101a, 101b, having housings 311a and 311b. Device 500a is similar to device 400 but for a controllable vent cover 375 provided in each of the housings 311a and 311b. Coupling of the connector body 350 with each of the two vial adapters 101a, 101b is similar to that of device 400 described above. Projecting from face 325 of the four-way connector body 350 is IV spike 322 shown with removable cap 102, IV spike 322 is configured for insertion into a liquid infusion container and has three lumens 323, 324a and 324b. Each of the two vial adapters 101a, 101b comprise a spike 200, each of the spikes comprising at least two lumen (e.g., fluid lumen 133a, 133b, and vent lumen 134a, 134b), each of the spikes configured for penetration of an container closure element, such as a pierceable septum of a medicament vial, and for providing fluid communication through device 500.

In one example, both housings 311a, 311b include anti-airlock feature (not shown), e.g., preventing airlock if fluid enters void space between vent filter 304 and vent lumen 134a, 134b. For example, if the vent becomes flooded, extending the orifice height with anti-airlock feature aids in evacuating the fluid in an inverted orientation. In another example, vent lumen 134a, 134b has its opening in spike 200 vertically transposed from opening in fluid lumen 133a, 133b, such that when device 100 is engaged with a container and inverted, the opening in vent lumen is above the liquid level of the container whereas the opening in vent lumen 134a, 134b is configured to be below the liquid level and configured to receive liquid adjacent the septum of the container such that a maximum amount of liquid can be transferred from the container through spike 200.

Vent body 305 can be constructed of a rigid plastic such as polycarbonate, polypropylene, cyclic olefinic copolymer, etc. In one example, vent body 305 is configured to receive a one-one valve 395 or a vent housing with a filter 304.

FIG. 10B is a section view along section line 10B-10B of FIG. 10A, showing housings 311a, 311b coupled to coupler body 350, providing fluidic communication independently between fluid lumen 133a, 133b of spikes 200, respectively and fluid lumen 324a, 324b of IV spike 322. Connector body 350 as shown is a four-way connector body comprising housing 311a including a first vial adapter 101a coupled to the connector body for receiving a media container with a stopper. First vial adapter 101a includes a first vial spike 200 with first fluid lumen 133a. A second vial adapter 101b is coupled to the connector body 350 also for receiving a media container with a stopper, the second vial adapter including a second vial spike 200. Second vial spike 200 has a second fluid lumen 133b. Device 500a, 500b also comprises an IV spike 323 integral with the connector body 350. IV spike 323 having a first IV fluid lumen 324a in direct fluid communication with the first fluid lumen 133a of spike 200 of vial adapter 101a. Second IV fluid lumen 324b of IV spike 323 is adjacent the first IV spike lumen 324a and the second IV fluid lumen 324a is in direct fluid communication with the second fluid lumen 133b of vial spike 200 of vial adapter 101b. IV spike has infusion lumen 323 in direct fluid communication with the connector body and the central fluid lumen 131.

This arrangement provides for fluidic path of fluid lumen 133a and fluid lumen 324a to be isolated from fluidic path of fluid lumen 133b and 324b. Moreover, fluid lumen 323 path to central lumen 141 is physically isolated from fluid lumen 324a, 324b, 133a and 133b paths.

Each vial adapter 101a, 101b is shown with female connectors 372, receivable by male connectors 382 of connector body 350, however, this configuration can be provided with the reverse (male/female) connector coupling. Each vial adapter 101a, 101b of device 500 can be either permanently or reversibly connected to connector body 350.

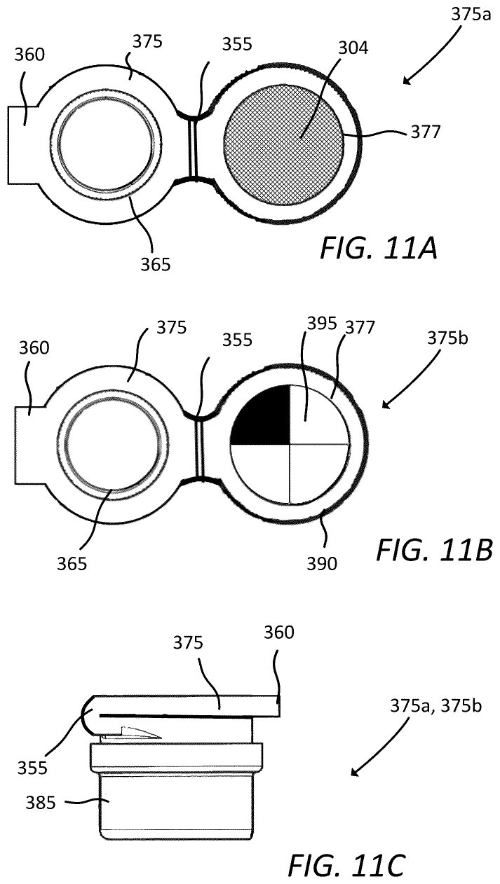

Vent body 305 has opening 307 in fluidic communication with vent lumens of spike 200, opening 307 configured to receive cover assembly 375a, 375b. As shown in FIGS. 11A, 11B, cover assembly 375a, 375b includes cover body 385 sized to be received by opening 307 of vent body 305. Cover body 385 defines opening 377 therethrough, one end 390 of cover body is coupled to cover 375 via hinge 355, shown in an open configuration. In one example, opening 377 is configured to receive filter 304 or one-way valve 395 and provide a hermetic seal therewith. In another example, opening 377 is configured to receive one-way valve 395 and provide a hermetic seal therewith without cover 375 or hinge 355, as illustrated in FIG. 8B. FIG. 11C depicts cover assembly 37a, 37b with cover 375 in a closed configuration with opening 377 providing a hermetic seal therewith.

In one example, cover assembly 375a, 375b is generally cylindrically shaped. In one example, cover 375 has annular wall 365 configured to be received by opening 377 of cover body 385 and provide a hermetic seal therewith. Cover body 385, cover 375, and annular wall 365 can be constructed of polyethylene, polyethylene copolymers, polypropylene, polypropylene copolymers, rubber, silicone, or thermoplastic elastomer.

In one example, cover 375, wall 365 and cover body 385 are constructed of the same material which is softer and more pliable (lower durometer) than that vent body 305, for example for press fitting, solvent bonding, etc., with opening 307 of vent body 305. Vent body 305, opening 307, 377, and wall 365 can be tapered.

Vent cover 375 is configured to transition from a first state, where wall 365 is in a leak-tight and airtight relationship with opening 377, to a second state, where wall 365 is dislodged from opening 377 such that a fluidic pathway is created between opening 377 and vent lumen 134a, 134b of spikes 200, respectively, through vent body 305. In one example, transition of vent cover 375 from the first state to the second state is reversible. In another example, transition of vent cover 375 from the first state to the second state is irreversible. In one example, cover 375 is completely removable from vent body 305. In another example, hinge 355 of vent cover 375 is configured to allow transition of vent cover 375 from the first state to the second state and to maintain a physical connection between the cover and the vent body 305. Tab feature 360 allows a user to easily transition vent cover 375 from the first state to the second state, for example, using a finger or thumb of one hand.

Vent body 305 of first housing 311a receives cover body 385. Opening 377 of cover body 385 receives wall 365 of vent cover 375 in the first state, providing a sealed relationship. Fluidic communication between vent lumen 134a, 134b and the ambient is controlled via vent cover 375, where in the first state fluidic access to ambient is sealed off through vent body 305 and vent lumens 134a, 134b of spikes 200, where in the second state, fluidic access to the ambient is provided through vent body 305 and opening 307 via vent lumens 134a, 134b of spikes 200.

Also shown with vent body 305 is optional vent filter 304. Vent filter 304 can consist of any appropriate material, micron porosity, hydrophobicity, and efficiency for a given application. A typical range of micron porosity for an vent filter is between 0.02 uM and 150 uM (micron). In one aspect, vent filter 304 is disk-shaped. Other shapes can be used for the vent filter 304.

Particulate may be present in the vial or become mixed in the medication during the fluid removal/transfer process. Types of particles that may be present in vials include;

pharmaceutical sediment, lipids, un-dissolved solids, crystallization of medication elements, glass, plastic and rubber debris, septum particulate and various other types of contaminants. These particles can be various sizes and shapes, and depending on the medicament, hydrophobic and/or hydrophilic, and/or partially or completely ionized salts. In addition, insertion of the vial access spike can shear off or core bits of the stopper of the vial. Often these particles are sub-visible sizes and will go unnoticed. Particulates that are injected into a patient can cause complications, for example, phlebitis, organ damage, and vessel blockage. This presents a technical problem of fluid filtering alone or in combination with the need for controlled venting.

Thus, a technical solution to the above technical problem is addressed by incorporating fluid filtering functionality as described in co-assigned U.S. Pat. No. 9,585,812, the disclosure of which is incorporated herein by reference, in device 100, 100′, 300 or 500.

### Administration Sets

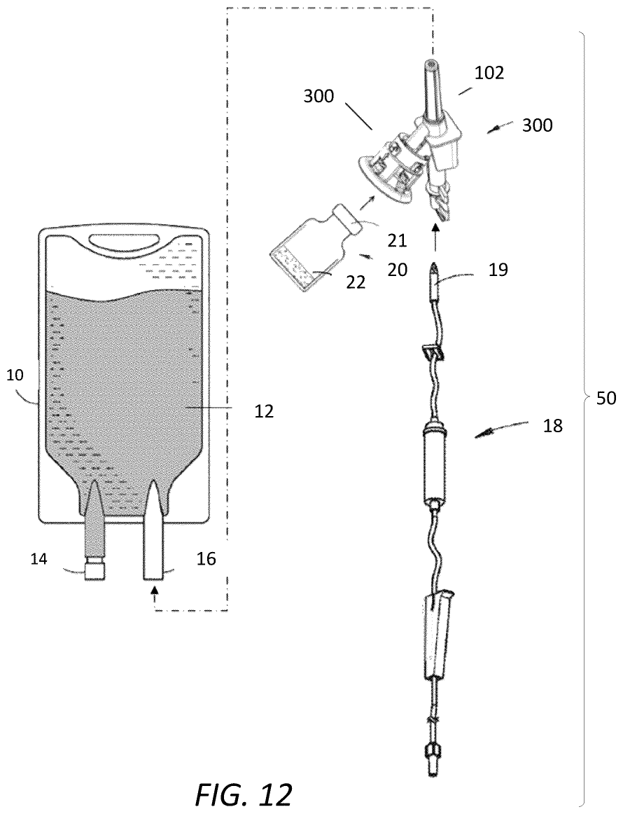

FIG. 12 depicts an administration set including an IV bag/infusion liquid bag, an exemplary transfer device 300 as disclosed and described in the present disclosure, a medicament/media vial or container, and an infusion set. Collectively, the presently disclosed device (100, 100′, 300), infusion liquid bag 10, media container 20 and a conventional infusion set 18 are used in combination, e.g., as kit 50. The use of the kit 50 is as follows: IV spike 122 is sealingly inserted into IV port 16 (or 14) and the vial adapter 101, 201, 301 is snap fitted on a media container 20 where vial spike penetrates media container stopper 21 for allowing introduction of the infusion liquid 12 of infusion liquid bag 10. Such adding is typically achieved by squeezing the infusion liquid bag 10 to urge infusion liquid 12 into the media container 20 and then inverting the infusion liquid bag 10 to facilitate transfer of the vial contents. When the media container 20 includes a lyophilized medicament 22, the infusion liquid would reconstitute the medicament and may include several cycles of urging infusion liquid into the media container 20 and draining vial contents to the IV bag 10. Infusion line spike 19 of the infusion set 18 is then sealingly inserted into the transfer device infusion port 143 after removal of twist-off closure member 144.

While not shown, the transfer device 100, 100′, 300 insofar as they are depicted with an IV port 143, such devices can be constructed with an integral infusion set instead of the IV port where tubing is directly connected to the IV port 143.

The use of the transfer device 300 is similar to the use of the transfer device 100, 100′, terms of adding medicament additive to the infusion liquid bag 10, but device 300 provides access control between vial 20 and infusion liquid bag 10 and/or infusion set 18. Thus, with reference to FIG. 3C, after connection of vial 20 with vial adapter 301, housing 210 can be rotated about longitudinal axis (co-aligned with section line 3D-3D). In a first configuration, device 300 has pathway 233 provided with access to spike lumen 133 for allowing fluid communication between device 300 and vial 20. In a second configuration, device 300 has pathway 233 misaligned with spike lumen 133 for blocking fluid communication between device 300 and vial 20.

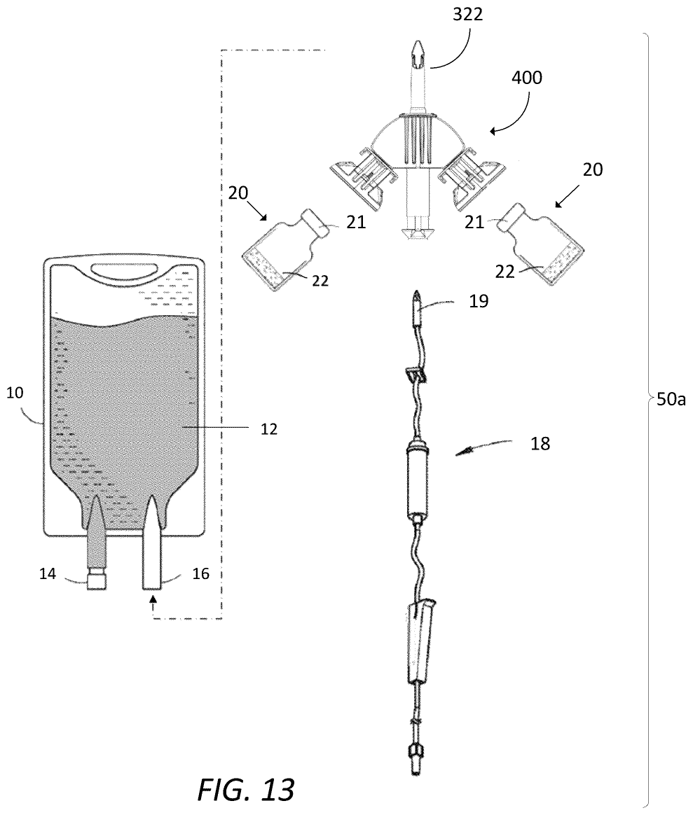

FIG. 13 depicts an administration set including an IV bag/infusion liquid bag, an exemplary dual container transfer device 400, multiple medicament/media vial or containers, and an infusion set. The use of the transfer device 400 is similar to the use of the transfer device 100, 100′, in terms of adding medicament additive to the infusion liquid bag 10, but device 400 provides an option for the user to use (access) one or both of the media containers 20, whereas each media container has releasable cover 202a, 202b having annual seat 75 sealing the corresponding vial spike 200a, 200b, and if one of the covers 202a, 202b is not removed, the device 400 functions as device 100, 100′ described above. In one example, device 400 provides for the use of containers 20 containing different medicaments and/or providing for a dual-drug dosing regimen. The presently disclosed device 400, infusion liquid bag 10, two media containers 20 and a conventional infusion set 18 are used in combination, e.g., as kit 50a. The use of the kit 50a is as follows: IV spike 322 is sealingly inserted into IV port 16 (or 14) and the vial adapter 101a and/or 101b is snap fitted on corresponding media containers 20 where corresponding vial spike 200a, 200b penetrates media container stoppers 21 for allowing introduction of the infusion liquid 12 of infusion liquid bag 10 into each container 20. Such adding is typically achieved by squeezing the infusion liquid bag 10 to urge infusion liquid 12 into the media container(s) 20 and then inverting the infusion liquid bag 10 to facilitate transfer of the vial(s) contents. When the media container 20 includes a lyophilized medicament, the infusion liquid would reconstitute the medicament and may include several cycles of urging infusion liquid into the media container 20 and draining vial contents to the IV bag 10. Infusion line spike 19 of the infusion set 18 is then sealingly inserted into the transfer device infusion port 143 after removal of twist-off closure member 144.

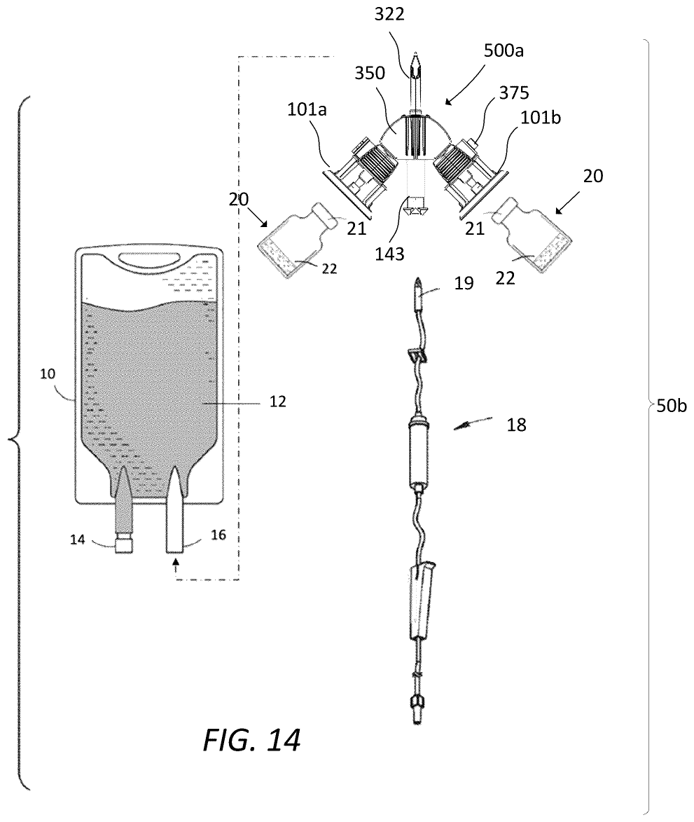

FIG. 14 depicts an administration set including an IV bag/infusion liquid bag, an exemplary dual container transfer device 500a with user controlled venting as disclosed and described in the present disclosure, multiple medicament/media vial or containers, and an infusion set. Device 500a, infusion liquid bag 10, two media containers 20 and a conventional infusion set 18 are used in combination, e.g., as kit 50b.

The use of the transfer device 500a and kit 50b is similar to the use of the transfer device 400 and kit 50a in terms of adding medicament additive to the infusion liquid bag 10, but device 500a provides venting independently between one or both of media containers 20 and infusion liquid bag 10 and/or infusion set 18. Thus, a method of transferring liquid between sealed vials and an infusion liquid container is provided as follows.

As shown in FIG. 14, a four-way connector body 350 has a first vial adapter 101a received by the connector body, the first vial adapter comprising a vent body 305 having a cover 375. Cover 375 of vial adapter 101a is transitionable from a hermetically sealed configuration with the corresponding vent body to an unsealed configuration. configured for reversibly hermetically sealing the vent body, and a spike 200 having a proximal end and a distal end, the spike having a fluid lumen 133a and a vent lumen 134a, the vent lumen in fluidic communication with the vent body. Cover 375 of adapter 101a is shown providing a hermetically sealed configuration with the vent body. Shroud 115 projects from the first vial adapter 101a and at least partially surrounds a portion of the spike 200, the shroud configured to receive a vial or container 20.

The four-way connector body 350 further receives a second vial adapter 101b comprising a vent body 305 having a cover 375. Cover 375 of vial adapter 101b is transitionable from a hermetically sealed configuration with the corresponding vent body to an unsealed configuration. Vial adapter 101b comprises a spike 200 having a fluid lumen 133b and a vent lumen 134b, the vent lumen in fluidic communication with the vent body. Shroud 115 projects from the second vial adapter 101b and at least partially surrounds a portion of the spike 200. Shroud 115 of adapter 101b is configured to receive an additional vial or container.

As shown, the cover 375 of adapter 101b is shown in an unsealed configuration with the vent body 305, however, one or both of cover 375 of first vial adapter or the second vial adapter can be in the hermetically sealed configuration with the corresponding vent body or the unsealed configuration.

The four-way connector body 350 further comprising an IV spike 322 integral with the connector body, the IV spike having an first IV fluid lumen 324a in fluidic communication with the fluid lumen 133a of the first vial adapter 101a, and a second IV fluid lumen 324b (adjacent the first IV fluid lumen) in direct fluid communication with the fluid lumen 133a of the second vial adapter 101b. vial adapter is snap fitted on corresponding media container 20 where vial spike 200 penetrates media container stoppers 21 for allowing introduction of the infusion liquid 12 of infusion liquid bag 10 into each container 20. Fluidic communication between the sealed vials and the first vial adapter and the second vial adapter is carried out and establishes fluidic communication between the IV spike 322 and the infusion liquid container 10, with the media container(s). In one example, at least one of the sealed vials comprises a medicament 22. In another example, the medicament 22 is reconstitutable (e.g., using the liquid 12 of infusion container 10) and at least one of the vials 20 is under reduced pressure.

Reconstitution is typically achieved by squeezing the infusion liquid bag 10 to urge infusion liquid 12 into the media container(s) 20 and then inverting the infusion liquid bag 10 to facilitate transfer of the vial(s) contents. When one or both media containers 20 includes a lyophilized medicament 22, the infusion liquid would reconstitute the medicament and may include several cycles of urging infusion liquid into the media container 20 and draining vial contents to the IV bag 10.

Manipulating the cover 375 (user-controlled) of one or both of the first vial adapter 101a or the second vial adapter 101b to the unsealed configuration with vent body cover provides venting of the system, either before or after the IV spike 322 is sealingly inserted into

IV port 16 (or 14). After or during sufficient reconstituting, mixing, or diluting of medicament has taken place, vent cover 375 is manipulated to an unsealed configuration, i.e., transitioned to the second state, opening a vent to allow for independent aspiration of either vial 20 via vent lumen 134a or 134b of spike 200, respectively, through vent body 305 with optional vent filter 304. In one example, only one vent cover 375 is opened. In another example, both vent covers 375 are opened. Infusion line spike 19 of the infusion set 18 is then sealingly inserted into the transfer device infusion port 143 after removal of twist-off closure member 144 (not shown) for drug delivery to a subject.

In one example, one or both media containers, e.g., vials 20 contains medicament held under reduced atmospheric pressure. In one example, reduced atmospheric pressure of vial 20 urges diluent of infusion liquid bag 10 to enter vial 20 for reconstituting, mixing, or diluting medicament contained therein.

While not shown, the transfer device 100, 100′, 300, 400, 500a and 500b insofar as they are depicted with an IV port 143, such devices can be constructed with an integral infusion set instead of the IV port where tubing is directly connected to the IV port 143.

While the disclosure has been described with respect to a limited number of embodiments, it will be appreciated that many variations, modifications, and other applications of the disclosure can be made within the scope of the appended claims.

We claim:

1. A transfer device, the transfer device comprising a three-way connector body having:
i) a vial adapter coupled to the three-way connector body for receiving a media container with a stopper, the vial adapter including a vial spike for fluidic communication therewith, the vial spike comprising at least one vial spike lumen, a distal annular edge connected to the vial adapter by four segments defining four openings, and four fingers positioned within the four segments;
ii) an IV spike integral with the three-way connector body, the IV spike having a first end for insertion into an infusion liquid container, and a second end for receiving an infusion port, a first IV lumen, and a second IV lumen adjacent the first IV lumen, the first IV lumen being in direct fluid communication with the at least one vial spike lumen, and the second IV lumen being in direct fluidic communication to the infusion port, through the three-way connector body, the first IV lumen and the second IV lumen being isolated from each other.

2. The transfer device of claim 1, wherein the second IV lumen is isolated from the first IV lumen throughout the three-way connector body.

3. The transfer device of claim 1, wherein the vial adapter is integral with the three-way connector body.

4. The transfer device of claim 1, wherein the vial adapter is releasably coupled with the three-way connector body.

5. The transfer device of claim 1, wherein the at least one vial spike lumen is configurable between:
a first configuration where the at least one vial spike lumen is in continuous fluid communication with the first IV lumen or second IV lumen; and
a second configuration, where, upon rotation of the vial adapter relative to the three-way connector body, the at least one vial spike lumen is in discontinuous fluid communication with the first IV lumen or the second IV lumen.

6. The transfer device of claim 1, wherein the at least one vial spike lumen has at least two physically separated vial lumens.

7. The transfer device of claim 6, wherein at least one of the at least two physically separated vial lumens is in direct fluid communication with at least one of a vent, a vent filter, a one-way valve, and a fluid filter.

8. The transfer device of claim 7, wherein the at least one of the at least two physically separated vial lumens are in direct fluid communication with the one-way check valve.

9. The transfer device of claim 8, wherein the one-way check valve is integral with the vial adapter.

10. The transfer device of claim 7, wherein the vent filter is hydrophobic.

11. The transfer device of claim 1, wherein the three-way connector body is coupled with an infusion set.

12. The transfer device of claim 1, further comprising an IV spike adapter coupled to the three-way connector body configured for receiving an IV set.
